(19) United States
(12) Patent Application Publication
(10) Pub. No.: US 2023/0398028 A1
(43) Pub. Date: Dec. 14, 2023

(54) Drive-Thru Medical Facility, And Systems And Methods Of Using Same

(71) Applicant: Viral Solutions, Decatur, GA (US)

(72) Inventors: Ronald Sanders JR., Decatur, GA (US); Benjamin Lefkove, Atlanta, GA (US); Bernard Coxton, Atlanta, GA (US)

(21) Appl. No.: 18/455,013

(22) Filed: Aug. 24, 2023

Related U.S. Application Data
(63) Continuation of application No. 18/174,222, filed on Feb. 24, 2023.
Application No. 18/174,222 is a continuation of application No. 63/313,544, filed on Feb. 24, 2022.

(51) Int. Cl. A61G 10/00; G16H 80/00

(52) U.S. Cl. CPC A61G 10/00; G16H 80/00

(57) ABSTRACT

A drive-thru medical facility having a vehicle pathway and a clinic building. The vehicle pathway has an entrance; at least one diagnosis bay positioned downstream of the entrance; an intermediate section positioned downstream of the at least one diagnosis bay; a first exit positioned downstream of the intermediate section; and at least one parking space, which can optionally extend from the intermediate section. Each diagnosis bay of the at least one diagnosis bay is configured to receive a respective vehicle. The clinic building is positioned to be accessible by a passenger of a vehicle parked within a parking space of the at least one parking space.

### CROSS-REFERENCE TO RELATED APPLICATION

This application is a continuation of U.S. patent application Ser. No. 18/174,222, which claims priority to and the benefit of the filing date of U.S. Provisional Patent Application No. 63/313,544, filed Feb. 24, 2022. Each of these applications is incorporated by reference herein in its entirety.

### FIELD

This disclosure is directed to drive-thru medical facilities and, in exemplary configurations, to drive-thru medical facilities that include separate vehicle pathways for patients who remain in their vehicle and patients who are required to park and enter into a building for further examination or diagnosis.

### BACKGROUND

Conventionally, medical facilities require patients to park their vehicles, enter a medical office building or hospital, check in with a receptionist, spend substantial time in a waiting area, enter an examination room when instructed, and spend additional time awaiting examination by a clinician before checking out with the receptionist. Often, patients are required to commit hours of time to such a visit, even when the actual medical examination can be completed within minutes.

Various approaches to drive-thru testing have been developed in response to the COVID-19 pandemic. However, these drive-thru testing sites are singularly focused on testing rather than comprehensive medical examination, and the testing sites do not include the medical equipment or medical examination rooms needed for more thorough on-site examination.

There is a need for medical facilities that provide for more efficient examination of patients. In particular, there is a need for medical facilities that are designed to permit quick, comprehensive examination of patients while also providing the infrastructure needed for more thorough examination of patients when required.

### SUMMARY

Disclosed herein is a drive-thru medical facility having a vehicle pathway and a clinic building. The vehicle pathway has an entrance, at least one diagnosis bay positioned downstream of the entrance, an intermediate section positioned downstream of the at least one diagnosis bay, a first exit positioned downstream of the intermediate section, and at least one parking space (e.g., a plurality of parking spaces), which can optionally extend from the intermediate section. Each diagnosis bay of the at least one diagnosis bay can be configured to receive a respective vehicle. The clinic building is positioned to be accessible by a passenger of a vehicle parked within a parking space of the plurality of parking spaces.

Also disclosed herein are systems and methods for using the disclosed drive-thru medical facilities.

Additional advantages of the disclosed system and method will be set forth in part in the description which follows, and in part will be understood from the description, or may be learned by practice of the disclosed system and method. The advantages of the disclosed system and method will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

FIG. 1 is a top schematic view of an exemplary drive-thru medical facility as disclosed herein.

FIG. 2 is a block diagram of an exemplary system including a patient interface, a computing device, and a clinic building as disclosed herein.

FIG. 3 is a block diagram of an exemplary computing device as disclosed herein.

FIG. 4 is a top schematic view of another exemplary drive-thru medical facility as disclosed herein.

FIG. 5 is a top schematic view of another exemplary drive-thru medical facility as disclosed herein.

FIG. 6 is a top schematic view of another exemplary drive-thru medical facility as disclosed herein.

FIG. 7 is a top schematic view of another exemplary drive-thru medical facility as disclosed herein.

FIG. 8 is a top schematic view of another exemplary drive-thru medical facility as disclosed herein.

FIG. 9 is a top schematic view of another exemplary drive-thru medical facility as disclosed herein.

FIG. 10A is a top schematic view of an exemplary drive-thru medical facility as disclosed herein. FIG. 10B is a close-up view of the buildings of the drive-thru medical facility of FIG. 10A.

FIGS. 11A, 11B, and 11C are side, front, and rear perspective views of an exemplary drive-thru medical facility as disclosed herein. FIG. 11D is a side perspective view of a an exemplary drive-thru medical facility having a canopy structure that extends over a portion of an outdoor waiting area.

FIGS. 12A-12B are front perspective views of an exemplary drive-thru medical facility as disclosed herein.

### DETAILED DESCRIPTION

The disclosed facilities, systems, and methods may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present disclosure which will be limited only by the appended claims.

It must be noted that as used herein and in the appended claims, the singular forms “a,” “an,” and “the” include plural references unless the context clearly dictates otherwise. Thus, for example, reference to “a diagnosis bay” includes one or more of such diagnosis bays, reference to “an exit path” includes one or more of such exit paths, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, up to 5%, or up to 1% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification. Thus, words denoting order, such as “first” or “next,” should be interpreted as optional aspects unless plain meaning or logic dictates otherwise.

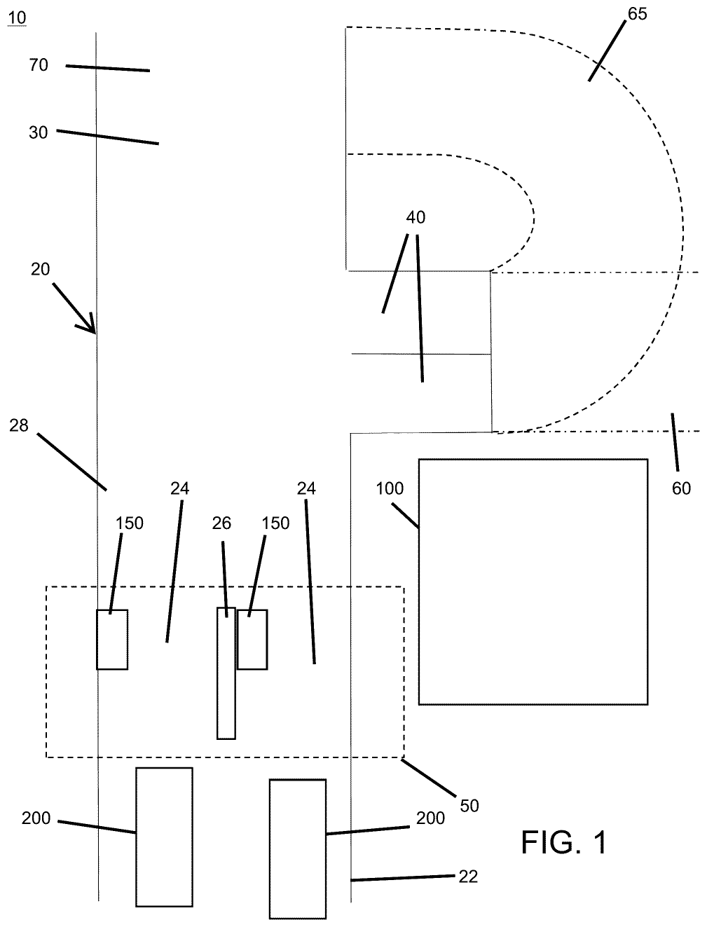

Disclosed herein and with reference to FIGS. 1-2 and 4-9, is a drive-thru medical facility comprising a vehicle pathway 20 and a clinic building 100. It is contemplated that the medical facility 10 can include only a single clinic building 100. However, in some aspects, the medical facility 10 can include a plurality of clinic buildings 100. As further disclosed herein, the vehicle pathway 20 can permit patients to receive an initial medical examination while the patients remain in their vehicles. Following the initial medical examination, each vehicle can proceed toward an exit of the facility, or the vehicle can proceed to a parking area that provides access to the clinic building 100. For a given patient, when the initial medical examination indicates that further examination is not required, the vehicle in which the patient is traveling can exit the facility. On the other hand, when the initial medical examination indicates that further examination is needed, the vehicle in which the patient is traveling can be parked, and the patient can then enter the clinic building for further medical examination. Following the further medical examination, the patient can return to the vehicle, and the vehicle can exit the facility (optionally, through a second exit path that is independent of and separate from a first exit path).

In various aspects, and with reference to FIG. 1, the vehicle pathway 20 can have an entrance 22 and at least one diagnosis bay 24 positioned downstream of the entrance. Each diagnosis bay 24 of the at least one diagnosis bay can be configured to receive a respective vehicle 200. As used herein, the term “diagnosis bay” should not be construed to require that particular conclusions be reached, or that particular steps be performed, at each bay; rather, the term “diagnosis bay” is meant to indicate that, in use as disclosed herein, each bay is located such that a patient positioned in a vehicle within the bay can receive a preliminary diagnosis or other indication of the patient's medical condition. Optionally, the at least one diagnosis bay can consist of a single diagnosis bay 24. However, in exemplary aspects, it is contemplated that the at least one diagnosis bay 24 can comprise a plurality of diagnosis bays. Optionally, in these aspects, it is contemplated that the plurality of diagnosis bays 24 can be oriented parallel or substantially parallel to one another. In further aspects, when the medical facility 10 comprises a plurality of diagnosis bays 24, the medical facility can further comprise at least one dividing structure 26. In these aspects, each dividing structure 26 of the at least one dividing structure can separate adjacent diagnosis bays of the plurality of diagnosis bays. In some exemplary aspects, it is contemplated that one or more of the diagnosis bays 24 can correspond to an area (e.g., a marked area) within the vehicle pathway to which vehicles are directed for examination of a patient within the vehicle.

Optionally, in still further aspects, it is contemplated that the medical facility 10 can further comprise at least one canopy structure 50 that at least partially overlies the at least one diagnosis bay 24. In these aspects, it is contemplated that the at least one canopy structure 50 can consist of a single canopy structure. In other aspects, it is contemplated that the at least one canopy structure 50 can comprise a plurality of canopy structures. In these aspects, it is contemplated that each canopy structure of the plurality of canopy structures can overlie at least a portion of a respective diagnosis bay. Optionally, the at least one canopy structure 50 can comprise a base portion (e.g., legs or support columns) that supports the canopy structure in an operative position above the at least one diagnosis bay 24. In some optional aspects, such a base portion can be at least partially associated with or coupled to a dividing structure 26, which can be securely positioned on an upper surface of the vehicle pathway 20. Optionally, it is contemplated that the at least one canopy structure 50 can comprise at least one solar panel that is configured to convert solar power into electrical power (for example, for use within the area of the diagnosis bay 24).

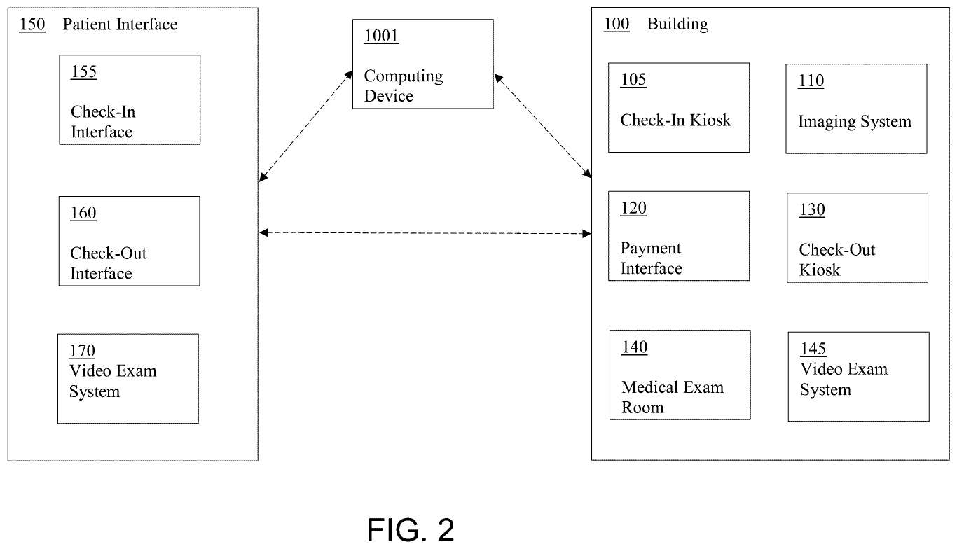

In exemplary aspects, and with reference to FIGS. 1-2, the medical facility 10 can further comprise at least one patient interface 150 that is positioned at or otherwise associated with the at least one diagnosis bay 24. Optionally, each diagnosis bay 24 can be equipped with a respective patient interface 150. Thus, when the at least one diagnosis bay 24 comprises a plurality of diagnosis bays 24, it is contemplated that the at least one patient interface 150 can comprise a plurality of patient interfaces 150, with each patient interface being positioned within or associated with a respective diagnosis bay 24. In exemplary aspects, the patient interface 150 can comprise a computing device (e.g., a portable computer, a tablet, a smartphone, a kiosk, and the like, or combinations thereof) that is configured to receive information from a patient within a vehicle. In some aspects, the patient interface 150 can be used by a facility operator (e.g., staff member) who is physically present at the at least one diagnosis bay 24. In these aspects, the facility operator can enter patient information (for example, information received in response to questions asked to the patient or other vehicle passengers) into the patient interface 150. Additionally, or alternatively, the patient interface 150 can be configured to permit direct entry of information by the patient (or other vehicle passenger) while the patient remains in the vehicle. For example, in some aspects, the patient interface 150 can be provided as a component of a kiosk that is positioned to permit communication between the patient interface and passengers of a vehicle while the vehicle is positioned within a given diagnosis bay 24. Optionally, in these aspects, the patient interface 150 can be located (optionally, fixedly positioned) on the left side (drivers' side) of each diagnosis bay.

In exemplary aspects, the patient interface 150 can comprise at least one processor and a memory that stores instructions that can be executed by the at least one processor to receive and/or deliver information to a patient through the patient interface 150. In these aspects, the instructions stored by the memory can be organized into a plurality of modules that are associated with specific functions. In various aspects, it is contemplated that the patient interface 150 can comprise a display and a user-input device as further disclosed herein. Optionally, in these aspects, the display can be a touch-screen display such that the display functions as the user-input device. Additionally, or alternatively, the user-input device can comprise a keypad, a keyboard, a microphone, a camera, a scanner, or the like.

In some aspects, and with reference to FIG. 2, the patient interface 150 can comprise a check-in interface 155 and a check-out interface 160. In these aspects, it is contemplated that the check-in interface 155 and the check-out interface 160 can be associated with separate, independent graphics and/or input forms that are provided at different times on the same (single) display of the patient interface 150. In other aspects, it is contemplated that the check-in interface 155 and the check-out interface 160 can be provided on separate, independent displays or on distinct portions of a single display.

In exemplary aspects, the check-in interface 155 can be positioned at the entrance, at the at least one diagnosis bay, or in between the entrance and the at least one diagnosis bay. In additional aspects, the check-out interface can be positioned at the diagnosis bay or downstream of the at least one diagnosis bay. Optionally, it is contemplated that the check-in interface 155 and/or the check-out interface 160 can be provided as or associated with a kiosk. In exemplary aspects, it is contemplated that the check-in interface 155 can be provided on a first (check-in) kiosk, while the check-out interface 160 can be provided on a second (check-out) kiosk that is positioned downstream of the check-in kiosk. In exemplary aspects, the check-in interface 155 can display a form for receiving information about a patient. Additionally, or alternatively, the check-in interface 155 can be configured to allow a user to access and load information associated with an existing patient account. For example, it is contemplated that the check-in interface can be configured to verify a user identify (for example, through password entry, multi-factor authentication, fingerprint scanning, eye-scanning, credit card verification, optical scanning of a code, combinations thereof, or other suitable authentication technique), and upon verification of the user identity, the check-in interface can be configured to retrieve and/or load existing patient information associated with the user account.

Optionally, in exemplary aspects, the efficiency of the medical facility 10 can be maximized through the use of a client application (an app) that can be downloaded by users (e.g., patients) and/or a web-based application that can be accessed on remote computing devices (e.g., smartphones, smart watches, tablets, and the like). In these aspects, it is contemplated that the client application and/or web-based application can allow users (e.g., patients) to remotely complete the check-in process before or upon arriving at the medical facility 10. In these aspects, upon verification of the user identity by the check-in interface 155, the information provided through the client application and/or web-based application can be retrieved and/or loaded into the check-in interface (or directly into a central computing device in communication with the check-in interface as further disclosed herein). Optionally, it is contemplated that the client application and/or web-based application can allow for creation of a patient account with which patient identifying information and data can be associated. Thus, in some aspects, upon verification of a patient identity by the check-in interface 155, the check-in interface can be configured to retrieve information and data from the patient account, thereby minimizing or avoiding the manual entry of information during the check-in process.

Thus, in operation, the check-in process can be performed using one or more of the following: the client application (using a smartphone or other remote computing device); the web-based application (using a smartphone or other remote computing device); a check-in interface 155 in the form of a computing device that receives inputs directly from a passenger in a vehicle (e.g., a kiosk); and/or a check-in interface 155 in the form of a computing device that receives input from a facility operator (e.g., a facility operator who has received requested information from a passenger of a vehicle).

After the patient identity has been confirmed and all requested information has been received, the patient interface 150 can be configured to provide an indication that the patient is ready for initial examination. Optionally, a facility operator who assisted with the check-in process can also perform the initial examination. Alternatively, it is contemplated that the patient interface 150 can be in communication with a central computing device 1001 as further disclosed herein that can provide an indication to a remote computing device that the patient is ready for the initial examination. In response to the indication received by the remote computing device, a clinician (for example, a clinician monitoring or responding to the remote computing device) can report (e.g., move) to the diagnosis bay where the patient is located. During the initial examination, the clinician can approach the vehicle at a location where the patient can be examined (e.g., through a window or door of the vehicle). It is contemplated that the initial examination can include questions asked by the clinician, a physical examination, and/or obtaining of one or more samples (e.g., blood, nasal and/or mouth swab, and the like).

Alternatively, or in addition to the in-person examination, the initial examination can include a remote examination performed using a video examination system 170 of the patient interface 150. In these aspects, it is contemplated that the video examination system 170 can comprise a display, a camera, and a microphone, which can optionally be provided as a component of a computing device of the patient interface 150. In exemplary aspects, the patient interface 150 can comprise a single display that sequentially provides the check-in interface 155, a video examination interface, and, optionally, the check-out interface 160. Alternatively, it is contemplated that the check-in interface 155 can be provided independently of the video examination interface. In use, the video examination system 170 can permit a clinician at a remote location to visually analyze and communicate with a patient.

After the initial examination is completed, the clinician (or team of clinicians, if appropriate) can determine if a more detailed examination of the patient is required. If a more detailed examination of the patient is required, then the patient can be directed to enter a clinic building 100 as further disclosed herein. If a more detailed examination of the patient is not required, then the patient can be directed to complete the facility visit by initiating the check-out process through the check-out interface 160. At this time, the clinician, through a computing device, can provide information concerning the condition and/or diagnosis of the patient, and the provided information can be associated with a patient account as further disclosed herein. During the check-out process, the patient can submit payment information (if required). Optionally, rather than submitting payment through the check-out interface 160, it is contemplated that payment can be submitted through the client application and/or web-based application on a smartphone or other remote computing device that is being operated by the patient (or other individual within the vehicle). After the check-out process is completed, the vehicle containing the patient can exit the facility as further disclosed herein.

As shown in FIG. 1, in further aspects, the vehicle pathway 20 can comprise an intermediate section 28 positioned downstream of the at least one diagnosis bay 24. In these aspects, it is contemplated that the intermediate section 28 can be configured to receive vehicles traveling through the at least one diagnosis bay 24. The vehicle pathway 20 can further comprise a first exit path 30 positioned downstream of (optionally, extending from) the intermediate section 28. The vehicle pathway 20 can still further comprise at least one parking space 40 (e.g., a plurality of parking spaces), which can optionally extend from the intermediate section 28. Optionally, the at least one parking space 40 can be separated from the first exit path 30. In use, it is contemplated that vehicles containing patients that do not require additional examination can exit the facility (optionally, by following the first exit path 30), whereas patients that require additional examination can park in one of the parking spaces 40 and then exit the vehicle for further examination (e.g., within a clinic building 100 as further disclosed herein).

Optionally, as shown in FIG. 1, the vehicle pathway 20 can further comprise an exit 70 that can be accessed by vehicles within the first exit path 30 and/or by vehicles exiting the at least one parking space 40. In some exemplary aspects, as shown in FIG. 1, the vehicle pathway can further comprise a connecting section 65 that is separate from the first exit path 30 and extends between the at least one parking space 40 and the exit 70. Thus, in these aspects, it is contemplated that a vehicle can enter a parking space 40 in a first direction, and the vehicle can exit the parking space by continuing to drive in the first direction to enter the connecting section After continuing to drive along the connecting section 65, the vehicle can enter the exit 70 (optionally, by merging with vehicles entering the exit from the first exit path). Thus, in some aspects, all vehicles that enter the facility through the entrance 22 can ultimately exit the facility through the exit 70, regardless of whether the vehicle parks in one of the parking spaces 40.

Optionally, as shown in FIG. 1, the at least one parking space 40 can define a portion of a second exit path 60 that is separate from the first exit path 30. In some aspects, each parking space 40 can be configured to permit a vehicle 200 in the intermediate section 28 to enter the parking space in a first direction, and the second exit path 60 can allow the vehicle to exit the parking space by driving in the first direction. In exemplary aspects, the second exit path 60 is separate from the exit 70. Thus, in these aspects, it is contemplated that vehicles that park in one of the parking spaces 40 can exit the facility at a different location than vehicles that do not park in one of the parking spaces (and simply proceed along the first exit path 30). Optionally, as shown in FIG. 1, the second exit path 60 can be oriented in a different direction than the first exit path 30.

In exemplary aspects, with reference to FIG. 1, the clinic building 100 can be positioned to be accessible by a passenger of a vehicle parked within a parking space 40. Optionally, in these aspects, the clinic building 100 can be positioned at least partially between the entrance 22 and the first exit path 30 of the vehicle pathway 20. In further aspects, it is contemplated that the clinic building 100 can be positioned on a first side of the parking space(s) 40, while the exit 70 is positioned on a second (opposite) side of the parking space(s) 40 (optionally, with the first and second sides spaced along an axis that is parallel to the direction of movement of the vehicles from the entrance 22 to the diagnosis bays 24.

In additional aspects, it is contemplated that the clinic building 100 can be configured for entry by patients who need additional or more detailed examination. Optionally, the clinic building 100 can comprise a check-in station (e.g., a check-in kiosk 105) where a patient entering the clinic building can confirm their identity. Optionally, the check-in kiosk can comprise a computing device that is configured to scan an identifier (e.g., a barcode, a QR-code, and the like) or to receive an input from the patient (e.g., a password or other identifying information). Upon completing the check-in process, the patient can receive further instruction (optionally, through visual instructions on a display associated with the check-in kiosk 105 and/or through audible instructions provided through a speaker associated with the check-in kiosk). For example, the patient can be directed to wait in a designated waiting area or to enter a specific room within the clinic building 100. Alternatively, rather than having a check-in kiosk 105, it is contemplated that the check-in station can comprise a location where a staff member confirms the identity of the patient and directs the patient within the building 100.

In some aspects, the clinic building can comprise a medical imaging system 110 (such as, for example, an X-ray machine, an ultrasound machine, a Magnetic Resonance Imaging (MRI) machine, and/or a Computed Tomography (CT) machine). Optionally, in these aspects, it is contemplated that such medical imaging systems can be positioned in designated rooms that are separated from other areas where patient examination may occur.

In further aspects, and with reference to FIG. 2, the clinic building can comprise at least one medical examination room 140 (optionally, a plurality of medical examination rooms). In these aspects, it is contemplated that a clinician can be available to conduct an in-person examination of a patient within a medical examination room 140. Thus, the medical examination room(s) 140 can comprise a patient examination table and other furniture (chairs) that may be needed for medical examination. The medical examination room(s) 140 can further comprise medical examination equipment, such as, for example and without limitation, one or more of the following: stethoscopes, blood pressure monitors, thermometers, pulse oximeters, blood drawing equipment, body weight scales, height rods, autoclave (or other sterilization equipment), a defibrillator, an audiometer, a centrifuge, a colposcope, an aspirator, an electrocardiogram (ECG) unit, laboratory diagnostic equipment (e.g., analyzer, a microscope, a specimen refrigerator/freezer), spirometer, fetal monitoring equipment, a holter monitor; resuscitation equipment, or oxygen supplementation equipment.

Following examination of a patient within the clinic building (optionally, within at least one of an examination room 140 or an imaging system 110), the patient can be directed toward a check-out station (e.g., a check-out kiosk 130). Optionally, the check-out station can comprise a payment interface 120 that is configured to accept and initiate processing of a payment from a user (on behalf of a patient). Optionally, the payment interface 120 can be associated with and displayed by the check-out kiosk 130. Alternatively, it is contemplated that the patient can be directed to provide payment using a client application or a web-based application that is operating on a remote computing device (e.g., a smartphone).

In conjunction with checking out (either through the patient interface 150 or at the clinic building 100), it is contemplated that the patient can receive additional instructions and/or follow-up information via the client application or the web-based application, which can be configured to allow for delivery of such instructions and/or information to an account profile associated with the patient. In this way, when the patient initiates the client application or the web-based application, the application can be configured to display the delivered instructions and/or information (for example, on a display of the remote computing device executing the client application or web-based application). Optionally, it is contemplated that the application can, using the remote computing device of a user, provide an audible, visual, and/or tactile indication of receipt of such instructions and/or information.

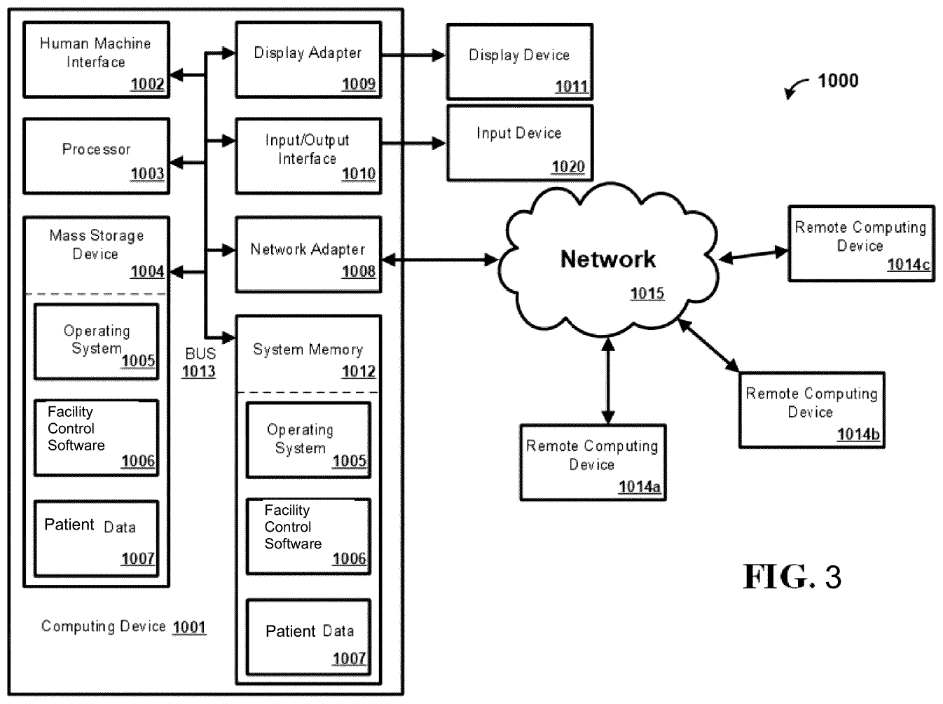

In exemplary aspects, and with reference to FIGS. 2-3, a system can comprise the drive-thru medical facility 10 and at least one computing device 1001 that comprises at least one processor and at least one memory in communication with the at least one processor. In these aspects, it is contemplated that the at least one processor can be configured to receive an input indicative of a diagnosis of a condition of a patient within a vehicle 200 positioned within a diagnosis bay 24. In these aspects, it is contemplated that the at least one computing device 1001 can be a central computing device that is in communication with computing devices associated with both the patient interface 150 and the clinic building 100. Additionally, or alternatively, it is contemplated that the at least one computing device 1001 that receives the input indicative of a diagnosis of a condition of the patient can be, or be associated with, the patient interface 150, and/or can be positioned within or otherwise associated with the clinic building 100.

In exemplary aspects, the at least one processor can be further configured to generate an invoice associated with examination of the patient. In these aspects, it is contemplated that the at least one computing device 1001 can be configured to directly display the invoice itself or to display the invoice using a display (e.g., screen) of one or more of the check-out interface 160, the check-out station (e.g., payment interface 120 and/or check-out kiosk 130) of the clinic building 100, the client application (executed by the computing device of a user), or the web-based application (executed by the computing device of a user). In still further aspects, the at least one processor can receive payment of the invoice, either directly or through payment information transmitted to the at least one processor by another computing device.

In further exemplary aspects, the at least one processor can be further configured to store, in the at least one memory, data indicative of the diagnosis of the condition of the patient. In these aspects, the stored data can be associated with an account of the user and configured for retrieval during future visits to the facility 10. It is further contemplated that portions of such data can be retrieved by the user via the client application and/or web-based application as further disclosed herein.

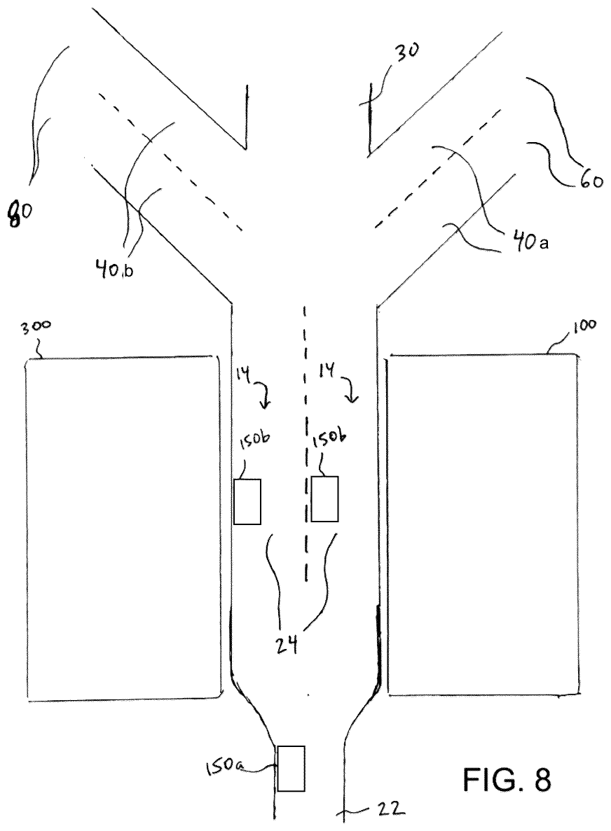

Optionally, in exemplary aspects, and with reference to FIG. 8, the clinic building 100 can be positioned on a first side of the at least one diagnosis bay 24. In these aspects, it is contemplated that the drive-thru medical facility can further comprise a second building 300 positioned on a second side of the at least one diagnosis bay 24 that is opposite the first side of the at least one diagnosis bay. In exemplary aspects, the second building 300 can be configured for use by members of the staff operating the facility (and not configured for patient entry or examination). For example, it is contemplated that the second building 300 can comprise one or more of: a locker room (comprising lockers), a rest room, a lounge area, a kitchen (with appliances), an office, a lab, or combinations thereof.

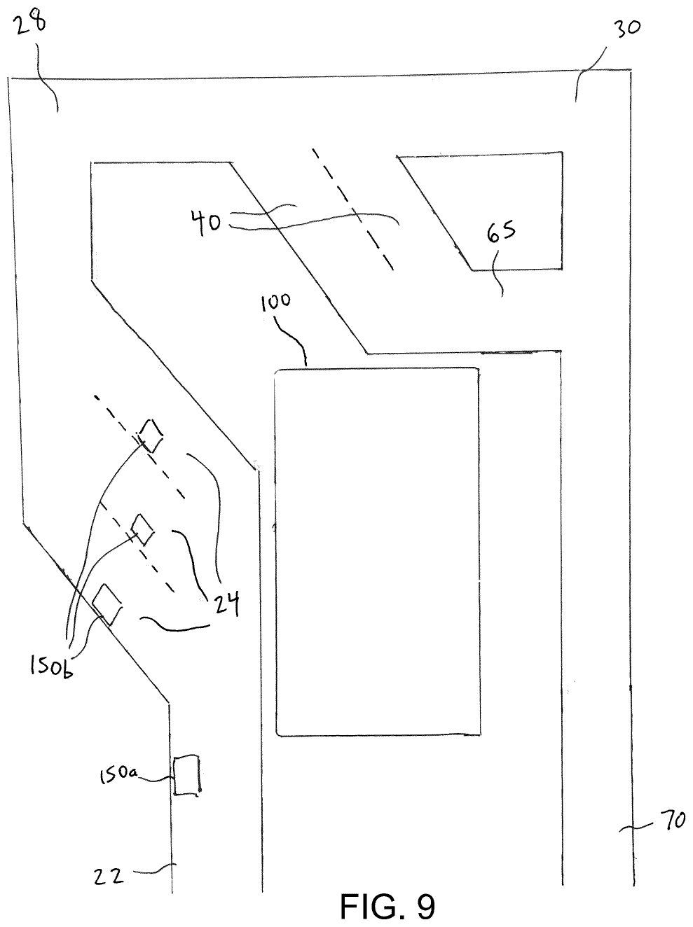

Optionally in exemplary aspects, and with reference to FIGS. 1 and 9, the entrance 22 can have a first axis, and the exit 70 can have a second axis that is parallel or substantially parallel to the first axis. For example, in some aspects, as shown in FIG. 9, the entrance 22 can be shaped to permit a vehicle to enter the vehicle pathway in a first direction along the first axis, and the exit 70 can be shaped to permit a vehicle to exit the vehicle pathway in a second direction along the second axis, with the first direction being opposite the second direction. In these aspects, it is contemplated that the clinic building 100 can be at least partially between the entrance and the exit of the vehicle pathway.

In further exemplary aspects, and with reference to FIG. 9, the at least one diagnosis bay 24 can be angularly oriented relative to the entrance 22 of the vehicle pathway.

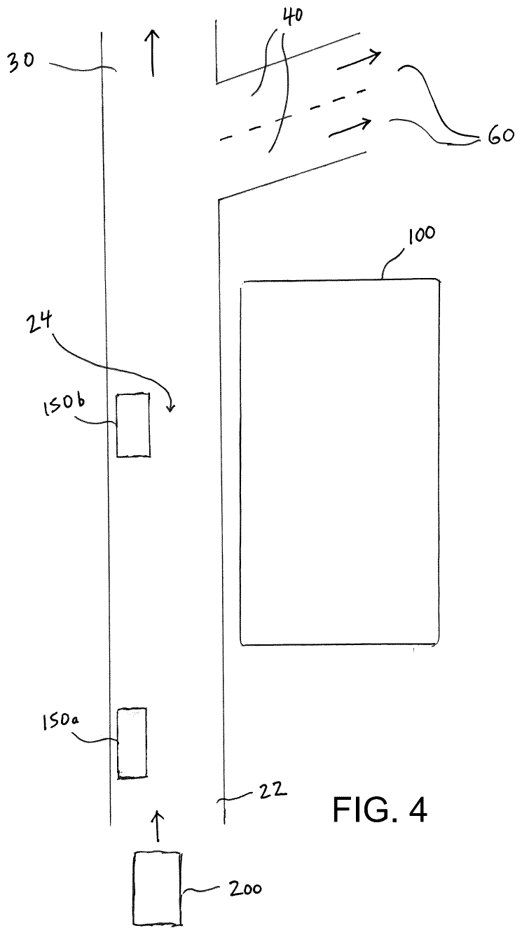

In further exemplary aspects, and with reference to FIGS. 4-9, the at least one parking space 40 can be angularly oriented relative to the intermediate section 28 of the vehicle pathway.

In further exemplary aspects, and with reference to FIG. 9, the at least one diagnosis bay 24 can extend along a bay axis, and the at least one parking space 40 can extend along a parking axis, with the bay axis being parallel or substantially parallel to the parking axis. Optionally, in these aspects, a portion of the intermediate section 28 of the vehicle pathway from which the at least one parking space 40 extends can be perpendicular or substantially perpendicular to the entrance 22 of the vehicle pathway.

In further exemplary aspects, and with reference to FIGS. 1 and 4-8, the vehicle pathway can comprise an exit 70 (e.g, a primary exit) that is (a) positioned to receive vehicles from within the first exit path 60 and/or the at least one parking space 40 and (b) in alignment or substantial alignment with the entrance 22. Optionally, in these aspects, the at least one diagnosis bay 24 can be parallel or substantially parallel to the exit 70 and the entrance 22.

In further exemplary aspects, and with reference to FIG. 8, the at least one parking space 40 can comprise a first plurality of parking spaces 40a that are positioned on a first side of the intermediate section 28 of the vehicle pathway, and the vehicle pathway can further comprise a second plurality of parking spaces 40b positioned on a second side of the intermediate section that is opposite the first side of the intermediate section. Optionally, in these aspects, the first plurality of parking spaces 40a can be angularly oriented relative to the intermediate section. Optionally, it is further contemplated that the second plurality of parking spaces 40b can be angularly oriented relative to the intermediate section. In exemplary aspects, the first and second pluralities of parking spaces 40a, 40b can be symmetrical about the intermediate section. For example, it is contemplated that the first and second pluralities of parking spaces 40a, 40b can be angularly oriented at the same or substantially the same angle with respect to the intermediate section (or other adjoining portion of the vehicle pathway).

In further exemplary aspects, and with reference to FIGS. 1 and 4-8, the vehicle pathway can comprise a second exit path 60 that includes the at least one parking space 40. In these aspects, each parking space of the at least one parking space 40 can be configured to permit a vehicle in the intermediate section 28 to enter the parking space in a first direction, and the second exit path 60 can allow the vehicle to exit the parking space by driving in the first direction. For example, in the embodiment shown in FIG. 8, the vehicle pathway can comprise a second exit path 60 that includes the first plurality of parking spaces 40a, and each parking space of the first plurality of parking spaces can be configured to permit a vehicle in the intermediate section to enter the parking space in a first direction, with the second exit path allowing the vehicle to exit the parking space by driving in the first direction. In this embodiment, the vehicle pathway can further comprise a third exit path 80 that includes the second plurality of parking spaces 40b, and each parking space of the second plurality of parking spaces can be configured to permit a vehicle in the intermediate section to enter the parking space in a second direction, with the third exit path allowing the vehicle to exit the parking space by driving in the second direction.

In some exemplary aspects, and with reference to FIGS. 4-5 and 7-9, rather than providing a single patient interface 150, it is contemplated that multiple patient interfaces 150a, 150b can be employed within a given vehicle pathway. For example, in some aspects, a first patient interface 150a can be positioned near the entrance 22 to permit patient check-in, and a second patient interface 150b can be positioned downstream of the first patient interface 150a (for example, positioned at a diagnosis bay 24) to permit examination and/or checkout.

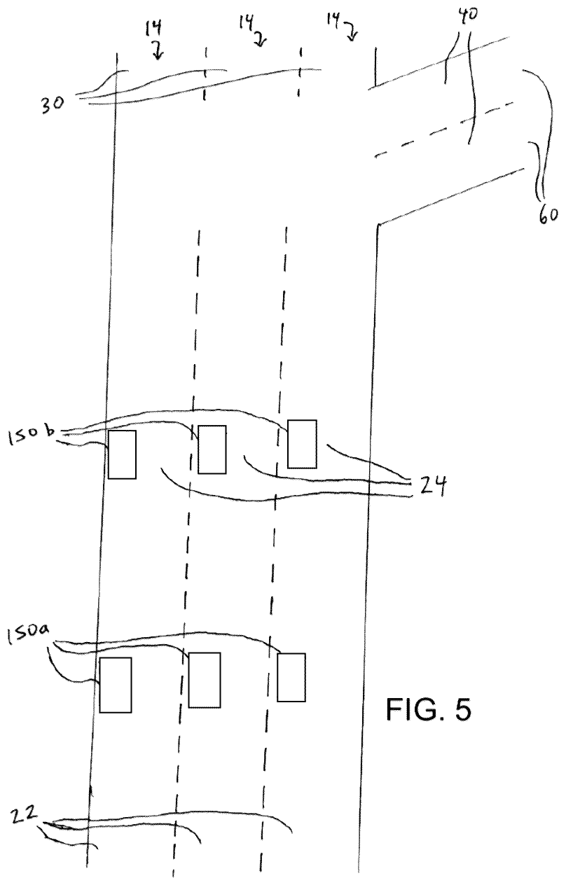

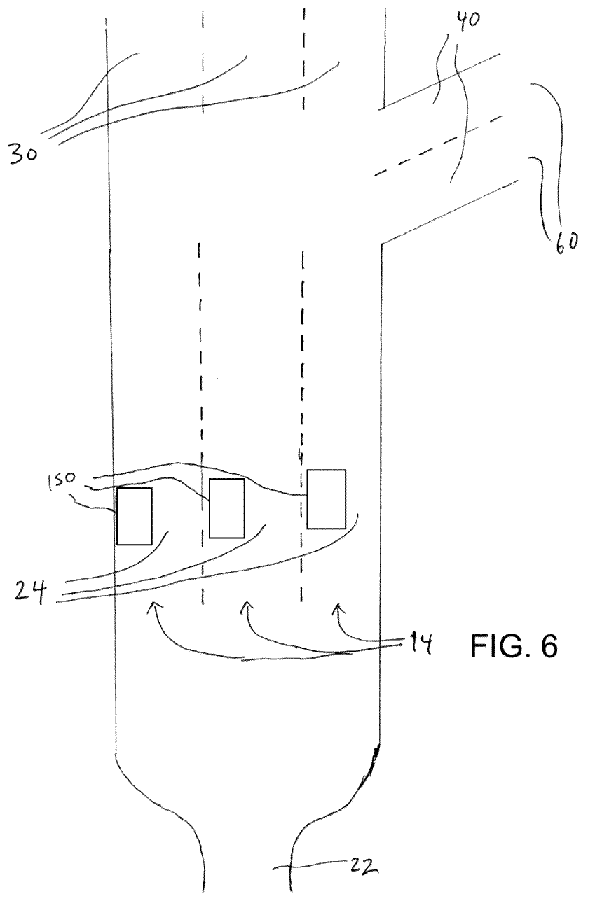

In some exemplary aspects, and with reference to FIGS. 5-6, the vehicle pathway can be divided into a plurality of lanes 14. For example, as shown in FIG. 5, a plurality of parallel lanes 14 can extend from the entrance 22 toward the first exit path 30. Thus, in this example, vehicles entering the facility 10 can enter the vehicle pathway by driving into a particular lane 14, and they can remain in the lane during the initial examination process. As another example, as shown in FIG. 6, the entrance 22 can comprise a single lane, and the vehicle pathway can widen downstream of the entrance 22 to form a plurality of parallel lanes 14.

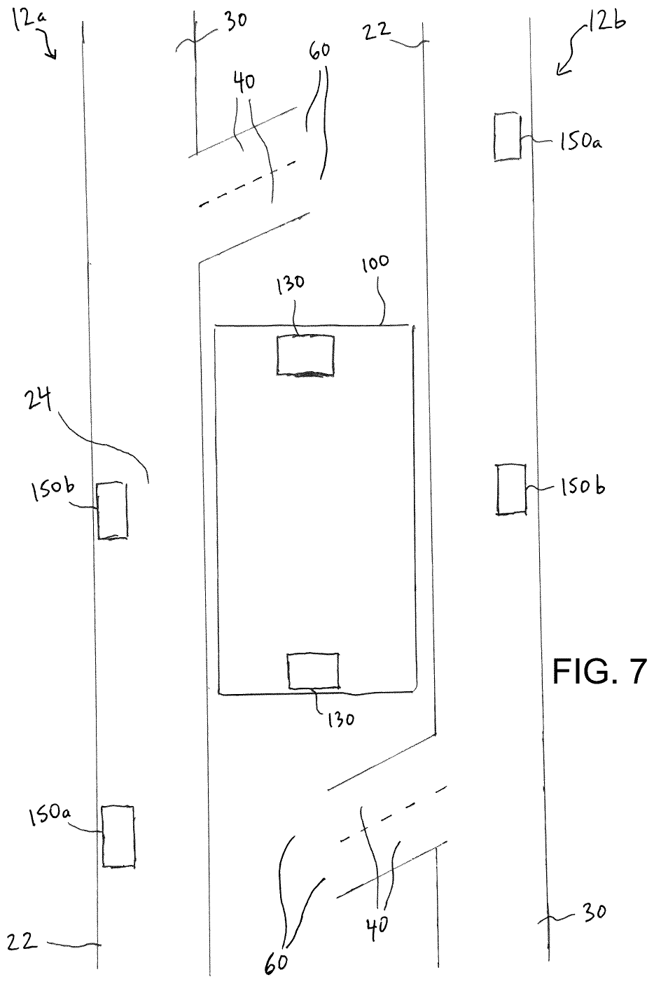

In some exemplary aspects, and with reference to FIG. 7, the facility can comprise two distinct vehicle pathways. For example, the facility can have two opposing lanes 12 positioned on opposite sides of the clinic building 100, with the direction of travel within the first lane 12 being opposite the direction of travel within the second lane. As shown in FIG. 7, it is contemplated that each vehicle pathway can further comprise at least one parking space 40 and its own second (separate) exit path 60. Optionally, the parking spaces of the two vehicle pathways can be positioned on opposing sides of the clinic building 100.

In use, it is contemplated that the various embodiments of the facility 10, including those shown in FIGS. 1 and 4-9, can be selected and implemented depending upon the particular location and conditions of the facility. For example, it is contemplated that the embodiment of FIG. 9 can be particularly suited to locations where vehicles can only enter and exit from one side of the property/location. As another example, it is contemplated that the embodiments that include multiple exit paths and/or multiple lanes can maximize the number of vehicles that can be accommodated within a facility, thereby avoiding or minimizing traffic difficulties.

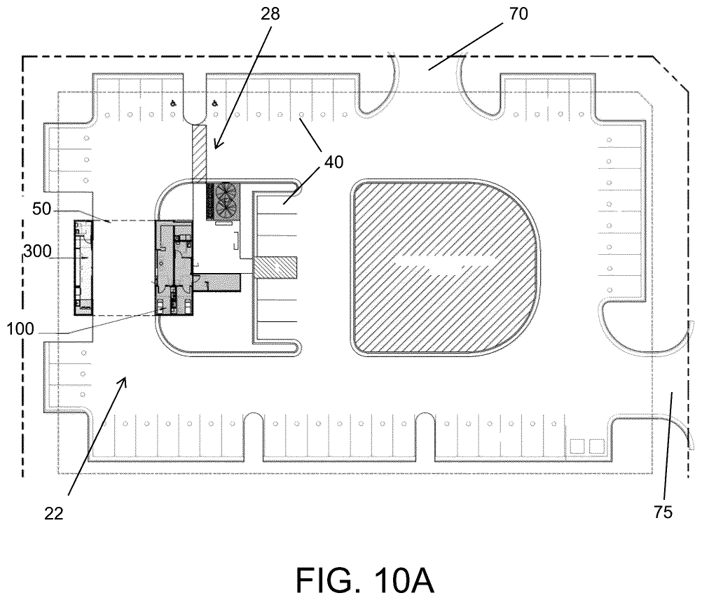

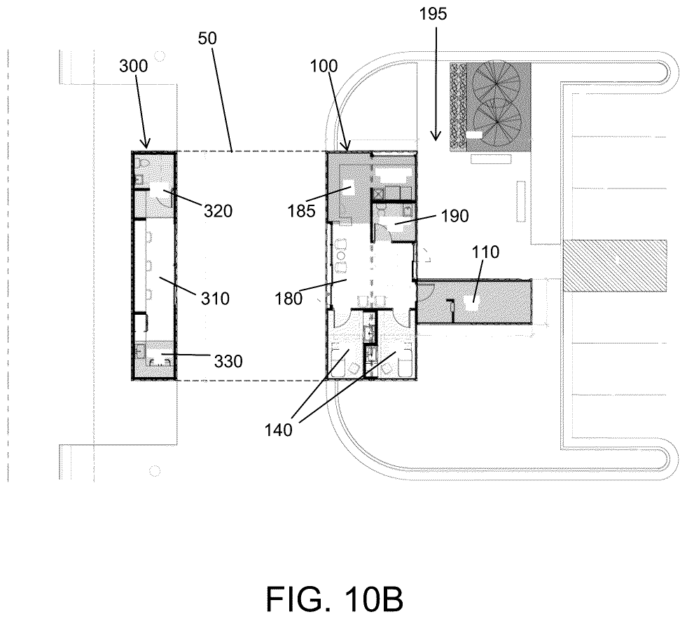

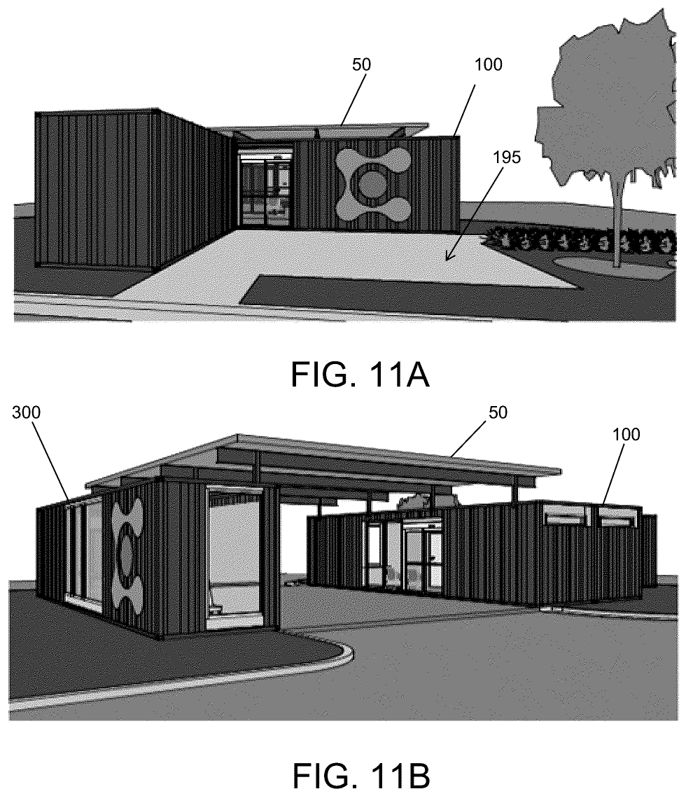

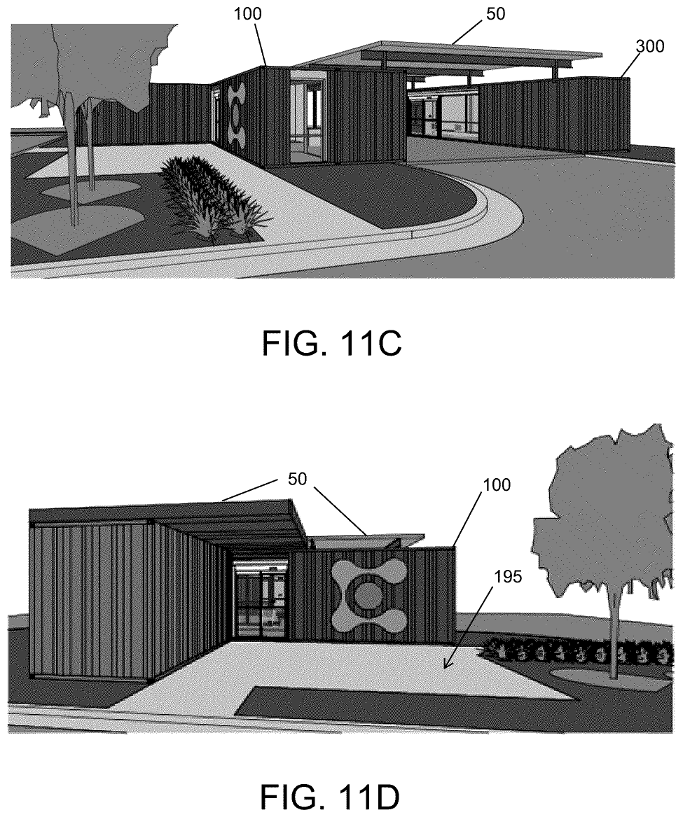

In various exemplary aspects, and with reference to the example embodiment depicted in FIGS. 10A-11D, it is contemplated that the facility 10 can be provided with a vehicle pathway that includes an entrance 22 and a canopy structure 50 that extends between a clinic building 100 and a second building 300. In exemplary aspects, supporting columns can extend upwardly from the clinic building 100 and the second building 300, and a cover panel that extends over the area between the clinic building and the second building can be supported by the supporting columns and/or by horizontal beams that are supported by the supporting columns. The diagnosis bay(s) 24 can be positioned within the portion of the vehicle pathway that underlies the canopy structure 50. Upon exiting the diagnosis bay(s) and traveling beyond the canopy structure 50, vehicles can enter the intermediate section 28, which can generally provide access to a standard parking lot, which includes parking spaces 40. The parking spaces 40 include ADA-compliant parking spaces in proximity to the clinic building 100. In addition to providing access to the parking spaces 40, the intermediate section 28 can provide access to one or more exits 70, 75, which can either be reached directly from within the intermediate section 28 or by following pathways within the parking lot that includes the parking spaces 40. In further aspects, rather than reaching the entrance 22 directly from a street, it is contemplated that a patient can reach the entrance 22 by first entering the parking lot area (for example, through the ingress/egress areas marked as exits 70, 75) and then traveling to the entrance 22.

Exemplary configurations of the clinic building 100 and the second building 300 are shown in FIG. 10B. As shown, the clinic building 100 can include an indoor lobby/waiting area 180, a laboratory space 185, a restroom 190, and at least one medical exam room 140 (optionally, at least first and second medical exam rooms). The clinic building 100 can further include an imaging system 110, which can be positioned within a separate room/section of the clinic building 100. In further aspects, the facility 10 can comprise an outdoor waiting area 195 that can be positioned outside an entrance to the indoor lobby 180. Optionally, as shown in FIG. 11D, the canopy structure 50 can include a canopy section (or a discrete canopy component) that overlies at least a portion of the outdoor waiting area 195, thereby shielding patients from precipitation and/or sun.

Optionally, as shown, the second building 300 can include a nurse station 310, which can optionally include equipment, supplies, and medications needed for the evaluation and diagnosis of patients as disclosed herein. The second building 300 can further include a break room 320 and/or a restroom 330 for facility workers to use.

Optionally, it is contemplated that the clinic building 100 and/or the second building 300 can be formed from a plurality of modular building structures, which can be pre-formed off-site and delivered to a location of the facility 10 during construction. For example, the clinic building 100 can comprise a first plurality of modular building structures (e.g., two rows of two modular structures that cooperatively define a rectangular structure) and at least one additional modular building structure that extends perpendicularly from the outer surface of one or more of the modular building structures of the first plurality of modular building structures. As shown in FIG. 10B, it is contemplated that the modular building structure that houses the imaging system 110 can extend perpendicularly from one or more modular building structures that define the lobby area of the clinic building 100. As shown in FIGS. 11A-11D, it is contemplated that the modular building structures can be formed with openings for doors or windows.

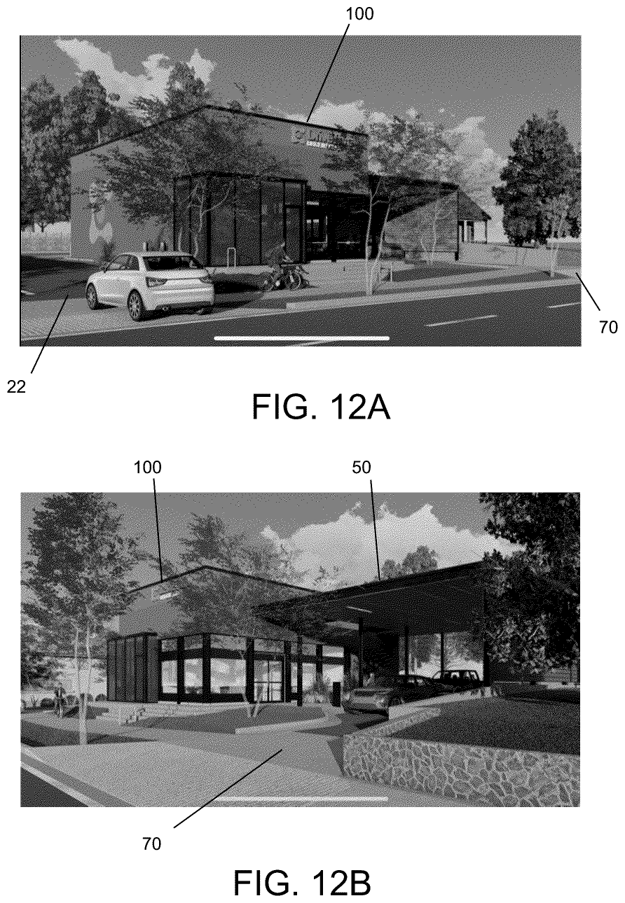

In various exemplary aspects, and with reference to the example embodiment depicted in FIGS. 12A-12B, it is contemplated that the facility 10 can be provided with a vehicle pathway that includes an entrance 22 and a canopy structure 50 that extends outwardly from a clinic building 100. In this example, vehicles can travel into the entrance 22 directly from a roadway, and the vehicle pathway can continue around the rear of the clinic building 100, at which point the vehicle can be directed underneath the canopy structure 50. Upon evaluation and diagnosis of a patient within the vehicle, if no additional examination is required, then the vehicle can return to the roadway through exit 70 and continue on their desired path of travel. On the other hand, if additional examination is required, then the vehicle can return to the roadway through exit 70 and then re-enter entrance 22 to access a parking space 40, which can be provided along the vehicle pathway between the entrance 22 and the canopy structure 50. After parking, the patient within the vehicle can enter the clinic building 100.

### Computing Devices

Except as where otherwise indicated, it is contemplated that any of the method steps described herein can be performed using one or more processors of one or more computing devices. It is further contemplated that any instructions provided to a patient (or other user of the facility) as disclosed herein can be performed by either a facility operator (e.g., staff member) or, alternatively, by a remote computing device in response to receipt of information by direct input or via sharing of information among the computing devices of the disclosed system.

FIG. 3 shows a control system 1000 including an exemplary configuration of a computing device 1001 for use within or in conjunction with operation of the facility 10. As shown in FIG. 2, it is contemplated that the computing device 1001 can be communicatively coupled to the patient interface 150 and/or computing devices positioned within the clinic building 100. Thus, in exemplary aspects, it is contemplated that the computing device 1001 can be configured to receive data from the patient interface 150 and/or clinic building 100. In further aspects, it is contemplated that the computing device 1001 can be configured to process data received from the patient interface 150 and/or clinic building 100 and, optionally, to provide instructions or data to the patient interface 150 and/or clinic building 100.

In some aspects, the computing device 1001 can be a single computing device that controls all aspects of the system 100. In further aspects, the computing device 1001 can be embodied by a plurality of computing devices that are in communication with each other on a network. For example, the computing device 1001 can comprise, or be embodied as, a tablet, smartphone, laptop, or desktop computer. In yet further aspects, various components of the facility 10 (e.g., the check-in interface(s), the check-out interface(s), the video examination system(s), imaging system, medical examination equipment, payment interface, etc.) can comprise respective controllers or computing devices that communicate with the computing device 1001 to perform various functions associated with or requested by the computing device 1001.

Although shown in FIG. 2 as being distinct from computing device 1001, it is contemplated that any one of the check-in interface 155, the check-out interface 160, the video examination system 170, the check-in kiosk 105, the imaging system 110, the payment interface 120, the check-out kiosk 130, equipment within the medical examination room 140, or the video examination system 145 can comprise a computing device having structure and/or features of computing device 1001, as shown in FIG. 3 and described further below.

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 may utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as patient data 1007 and/or program modules such as operating system 1005 and facility control software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and facility control software 1006 may be stored on the mass storage device 1004. One or more of the operating system 1005 and facility control software 1006 (or some combination thereof) may comprise program modules and the facility control software 1006. The patient data 1007 may also be stored on the mass storage device 1004. The patient data 1007 may be stored in any of one or more databases known in the art. The databases may be centralized or distributed across multiple locations within the network 1015.

A user may enter commands and information into the computing device 1001 using an input device (not shown). Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices. The display 1011 can show one or more outputs indicative of system progress, for example.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the control system 1000 can be configured with internet connectivity.

Thus, in exemplary aspects, it is contemplated that users of the facility 10 can communicate with computing device 1001 (and other computing devices within the facility) using a remote computing device 1014a,b,c as disclosed herein. For example, in some aspects, each user of the facility 10 can use a respective remote computing device (e.g., smartphone or tablet) to execute a client application or web-based application that allows for communication with the various computing devices associated with the facility. Such communication can occur during the user's visit to the facility 10 (for example, to provide information in response to various requests (e.g., payment information, medical information, patient identification, etc.)), as well as before the visit (e.g., to provide medical information and other information requested at check in) and/or after the visit (e.g., to provide updated information concerning patient condition and/or to permit delivery of further instructions or information from a clinician).

### Exemplary Aspects

In view of the described devices, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A drive-thru medical facility comprising: a vehicle pathway having: an entrance; at least one diagnosis bay positioned downstream of the entrance, each diagnosis bay of the at least one diagnosis bay being configured to receive a respective vehicle; an intermediate section positioned downstream of the at least one diagnosis bay; a first exit path extending from the intermediate section; and at least one parking space extending from the intermediate section and separated from the first exit path; and a clinic building, wherein the clinic building is positioned to be accessible by a passenger of a vehicle parked within a parking space of the at least one parking space.

Aspect 2: The drive-thru medical facility of aspect 1, wherein the at least one diagnosis bay comprises a plurality of diagnosis bays.

Aspect 3: The drive-thru medical facility of aspect 2, wherein the plurality of diagnosis bays are oriented parallel or substantially parallel to one another.

Aspect 4: The drive-thru medical facility of aspect 2 or aspect 3, further comprising at least one dividing structure, wherein each dividing structure of the at least one dividing structure separates adjacent diagnosis bays of the plurality of diagnosis bays.

Aspect 5: The drive-thru medical facility of any one of the preceding aspects, further comprising at least one canopy structure that at least partially overlies the at least one diagnosis bay.

Aspect 6: The drive-thru medical facility of any one of the preceding aspects, wherein the at least one parking space defines a portion of a second exit path that is separate from the first exit path.

Aspect 7: The drive-thru medical facility of any one of the preceding aspects, wherein the clinic building is at least partially between the entrance and the first exit path of the vehicle pathway.

Aspect 8: The drive-thru medical facility of any one of aspects 1-5 or 7, wherein the vehicle pathway further comprises an exit in communication with the first exit path and the at least one parking space.

Aspect 9: The drive-thru medical facility of aspect 6, wherein each parking space of the at least one parking space is configured to permit a vehicle in the intermediate section to enter the parking space in a first direction, wherein the second exit path allows the vehicle to exit the parking space by driving in the first direction.

Aspect 10: The drive-thru medical facility of any one of the preceding aspects, wherein the clinic building comprises a medical imaging system.

Aspect 11: The drive-thru medical facility of any one of the preceding aspects, wherein the clinic building comprises a payment interface.

Aspect 12: The drive-thru medical facility of aspect 11, wherein the payment interface comprises a check-out kiosk.

Aspect 13: The drive-thru medical facility of any one of the preceding aspects, wherein the clinic building comprises a medical examination room.

Aspect 14: The drive-thru medical facility of any one of the preceding aspects, further comprising a check-in kiosk positioned at the entrance, at the at least one diagnosis bay, or in between the entrance and the at least one diagnosis bay.

Aspect 15: The drive-thru medical facility of any one of the preceding aspects, further comprising a check-out kiosk positioned at the at least one diagnosis bay.

Aspect 16: The drive-thru medical facility of any one of the preceding aspects, further comprising a video examination system positioned at the at least one diagnosis bay.

Aspect 17: The drive-thru medical facility of aspect 5, wherein the at least one canopy structure comprises a plurality of canopy structures, and wherein each diagnosis bay of the plurality of diagnosis bays comprises a respective canopy structure of the plurality of canopy structures.

Aspect 18: The drive-thru medical facility of any one of the preceding aspects, wherein the clinic building is positioned on a first side of the at least one diagnosis bay, wherein the drive-thru medical facility further comprises a second building positioned on a second side of the at least one diagnosis bay that is opposite the first side of the at least one diagnosis bay.

Aspect 19: The drive-thru medical facility of aspect 5 or aspect 17, wherein the at least one canopy structure comprises at least one solar panel.

Aspect 20: The drive-thru medical facility of aspect 8, wherein the entrance has a first axis, and wherein the exit has a second axis that is parallel or substantially parallel to the first axis.

Aspect 21: The drive-thru medical facility of aspect 20, wherein the entrance is shaped to permit a vehicle to enter the vehicle pathway in a first direction along the first axis, and wherein the exit is shaped to permit a vehicle to exit the vehicle pathway in a second direction along the second axis, wherein the first direction is opposite the second direction.

Aspect 22: The drive-thru medical facility of any one of aspects 20-21, wherein the clinic building is at least partially between the entrance and the exit of the vehicle pathway.

Aspect 23: The drive-thru medical facility of any one of the preceding aspects, wherein the at least one diagnosis bay is angularly oriented relative to the entrance of the vehicle pathway.

Aspect 24: The drive-thru medical facility of any one of the preceding aspects, wherein the at least one parking space is angularly oriented relative to the intermediate section of the vehicle pathway.

Aspect 25: The drive-thru medical facility of any one of the preceding aspects, wherein the at least one diagnosis bay extends along a bay axis, wherein the at least one parking space extends along a parking axis, and wherein the bay axis is parallel or substantially parallel to the parking axis.

Aspect 26: The drive-thru medical facility of aspect 25, wherein a portion of the intermediate section of the vehicle pathway from which the plurality of parking spaces extend is perpendicular or substantially perpendicular to the entrance of the vehicle pathway.

Aspect 27: The drive-thru medical facility of aspect 6, wherein the vehicle pathway further comprises an exit in communication with the first exit path and the at least one parking space.

Aspect 28: The drive-thru medical facility of aspect 27, wherein the exit is in alignment with the entrance.

Aspect 29: The drive-thru medical facility of aspect 27 or aspect 28, wherein the at least one diagnosis bay is parallel or substantially parallel to the exit and the entrance.

Aspect 30: The drive-thru medical facility of any one of aspects 27-29, wherein the at least one parking space comprises a first plurality of parking spaces, wherein the first plurality of parking spaces is positioned on a first side of the intermediate section of the vehicle pathway, and wherein the vehicle pathway further comprises a second plurality of parking spaces positioned on a second side of the intermediate section that is opposite the first side of the intermediate section.

Aspect 31: The drive-thru medical facility of aspect 30, wherein the first plurality of parking spaces is angularly oriented relative to the intermediate section.

Aspect 32: The drive-thru medical facility of aspect 30 or aspect 31, wherein the second plurality of parking spaces is angularly oriented relative to the intermediate section.

Aspect 33: The drive-thru medical facility of any one of aspects 27-29, wherein the vehicle pathway comprises a second exit path that includes the at least one parking space, wherein each parking space of the at least one parking space is configured to permit a vehicle in the intermediate section to enter the parking space in a first direction, wherein the second exit path allows the vehicle to exit the parking space by driving in the first direction.

Aspect 34: The drive-thru medical facility of any one of aspects 30-32, wherein the vehicle pathway comprises a second exit path that includes the first plurality of parking spaces, wherein each parking space of the first plurality of parking spaces is configured to permit a vehicle in the intermediate section to enter the parking space in a first direction, wherein the second exit path allows the vehicle to exit the parking space by driving in the first direction.

Aspect 35: The drive-thru medical facility of any one of aspects 30-32 or 34, wherein the vehicle pathway comprises a third exit path that includes the second plurality of parking spaces, wherein each parking space of the second plurality of parking spaces is configured to permit a vehicle in the intermediate section to enter the parking space in a second direction, wherein the third exit path allows the vehicle to exit the parking space by driving in the second direction.

Aspect 36: A system comprising: the drive-thru medical facility of any one of the preceding aspects; and at least one computing device, wherein the at least one computing device comprises at least one processor and at least one memory in communication with the at least one processor, wherein the at least one processor is configured to: receive an input indicative of a diagnosis of a condition of a patient within a vehicle positioned within a diagnosis bay of the at least one diagnosis bay.

Aspect 37: The system of aspect 36, wherein the at least one processor is further configured to: generate an invoice associated with examination of the patient; and receive payment of the invoice.

Aspect 38: The system of aspect 36 or aspect 37, wherein the at least one processor is further configured to store, in the at least one memory, data indicative of the diagnosis of the condition of the patient, wherein the stored data is associated with an account of the user.

Aspect 39: A method of using the drive-thru medical facility of any one of aspects 1-35, the method comprising: receiving, by at least one computing device, an input indicative of a diagnosis of a condition of a patient within a vehicle positioned within a diagnosis bay of the at least one diagnosis bay.

Aspect 40: The method of aspect 39, further comprising: evaluating one or more physical properties of the patient; and providing, to the computing device, the input indicative of the diagnosis of the condition of the patient.

Aspect 41: The method of aspect 39 or aspect 40, further comprising: conducting further evaluation of the patient within the clinic building.

Aspect 42: The method of aspect 41, wherein conducting further evaluation of the patient comprises imaging of at least a portion of a body of the patient.

Aspect 43: The method of any one of aspects 39-42, further comprising receiving, by the at least one computing device, payment associated with evaluation and diagnosis of the condition of the patient.

Aspect 1A: A drive-thru medical facility comprising: a vehicle pathway having: an entrance; at least one diagnosis bay positioned downstream of the entrance, each diagnosis bay of the at least one diagnosis bay being configured to receive a respective vehicle; an intermediate section positioned downstream of the at least one diagnosis bay, wherein the intermediate section receives each vehicle traveling through a diagnosis bay along the vehicle pathway; a first exit positioned downstream of the intermediate section; and at least one parking space; and a clinic building, wherein the clinic building is positioned to be accessible by a passenger of a vehicle parked within a parking space of the at least one parking space.

Aspect 2A: The drive-thru medical facility of aspect 1A, wherein the at least one diagnosis bay comprises a plurality of diagnosis bays.

Aspect 3A: The drive-thru medical facility of any one of aspects 1A-2A, wherein the at least one parking space extends from the intermediate section.

Aspect 4A: The drive-thru medical facility of any one of aspects 1A-3A, further comprising at least one canopy structure that at least partially overlies the at least one diagnosis bay.

Aspect 5A: The drive-thru medical facility of any one of aspects 1A-4A, wherein the clinic building is at least partially between the entrance and the first exit of the vehicle pathway.

Aspect 6A: The drive-thru medical facility of any one of aspects 1A-5A, wherein the clinic building comprises a medical imaging system.

Aspect 7A: The drive-thru medical facility of any one of aspects 1A-6A, wherein the clinic building comprises a payment interface.

Aspect 8A: The drive-thru medical facility of aspect 7A, wherein the payment interface comprises a check-out kiosk.

Aspect 9A: The drive-thru medical facility of any one of aspects 1A-8A, wherein the clinic building comprises a medical examination room.

Aspect 10A: The drive-thru medical facility of any one of aspects 1A-9A, further comprising a check-in kiosk positioned at the entrance, at the at least one diagnosis bay, or in between the entrance and the at least one diagnosis bay.

Aspect 11A: The drive-thru medical facility of any one of aspects 1A-10A, further comprising a check-out kiosk positioned at the at least one diagnosis bay.

Aspect 12A: The drive-thru medical facility of any one of aspects 1A-10A, further comprising a video examination system positioned at the at least one diagnosis bay.

Aspect 13A: The drive-thru medical facility of any one of aspects 1A-12A, wherein the clinic building is positioned on a first side of the at least one diagnosis bay, wherein the drive-thru medical facility further comprises a second building positioned on a second side of the at least one diagnosis bay that is opposite the first side of the at least one diagnosis bay.

Aspect 14A: A system comprising: the drive-thru medical facility according to any one of aspects 1A-13A; and at least one computing device, wherein the at least one computing device comprises at least one processor and at least one memory in communication with the at least one processor, wherein the at least one processor is configured to: receive an input indicative of a diagnosis of a condition of a patient within a vehicle positioned within a diagnosis bay of the at least one diagnosis bay.

Aspect 15A: The system of aspect 14A, wherein the at least one processor is further configured to: generate an invoice associated with examination of the patient; and receive payment of the invoice.

Aspect 16A: The system of aspect 14A or aspect 15A, wherein the at least one processor is further configured to store, in the at least one memory, data indicative of the diagnosis of the condition of the patient, wherein the stored data is associated with an account of the user.

Aspect 17A: A method of using the drive-thru medical facility of any one of aspects 1A-13A, the method comprising: receiving, by at least one computing device, an input indicative of a diagnosis of a condition of a patient within a vehicle positioned within a diagnosis bay of the at least one diagnosis bay.

Aspect 18A: The method of aspect 17A, further comprising: evaluating one or more physical properties of the patient; and providing, to the computing device, the input indicative of the diagnosis of the condition of the patient.

Aspect 19A: The method of aspect 18A, further comprising: conducting further evaluation of the patient within the clinic building.

Aspect 20A: The method of aspect 18A or aspect 19A, further comprising receiving, by the at least one computing device, payment associated with evaluation and diagnosis of the condition of the patient.

Although several embodiments of the invention have been disclosed in the foregoing specification, it is understood by those skilled in the art that many modifications and other embodiments of the invention will come to mind to which the invention pertains, having the benefit of the teaching presented in the foregoing description and associated drawings. It is thus understood that the invention is not limited to the specific embodiments disclosed herein, and that many modifications and other embodiments are intended to be included within the scope of the appended claims. Moreover, although specific terms are employed herein, as well as in the claims which follow, they are used only in a generic and descriptive sense, and not for the purposes of limiting the described invention, nor the claims which follow.

What is claimed is:

1. A drive-thru medical facility comprising:
a vehicle pathway having:
an entrance;
at least one diagnosis bay positioned downstream of the entrance, each diagnosis bay of the at least one diagnosis bay being configured to receive a respective vehicle;
an intermediate section positioned downstream of the at least one diagnosis bay, wherein the intermediate section receives each vehicle traveling through a diagnosis bay along the vehicle pathway;
a first exit positioned downstream of the intermediate section; and
at least one parking space; and
a clinic building, wherein the clinic building is positioned to be accessible by a passenger of a vehicle parked within a parking space of the at least one parking space.

2. The drive-thru medical facility of claim 1, wherein the at least one diagnosis bay comprises a plurality of diagnosis bays.

3. The drive-thru medical facility of claim 1, wherein the at least one parking space extends from the intermediate section.

4. The drive-thru medical facility of claim 1, further comprising at least one canopy structure that at least partially overlies the at least one diagnosis bay.

5. The drive-thru medical facility of claim 1, wherein the clinic building is at least partially between the entrance and the first exit of the vehicle pathway.

6. The drive-thru medical facility of claim 1, wherein the clinic building comprises a medical imaging system.

7. The drive-thru medical facility of claim 1, wherein the clinic building comprises a payment interface.

8. The drive-thru medical facility of claim 7, wherein the payment interface comprises a check-out kiosk.

9. The drive-thru medical facility of claim 1, wherein the clinic building comprises a medical examination room.

10. The drive-thru medical facility of claim 1, further comprising a check-in kiosk positioned at the entrance, at the at least one diagnosis bay, or in between the entrance and the at least one diagnosis bay.

11. The drive-thru medical facility of claim 1, further comprising a check-out kiosk positioned at the at least one diagnosis bay.

12. The drive-thru medical facility of claim 1, further comprising a video examination system positioned at the at least one diagnosis bay.

13. The drive-thru medical facility of claim 1, wherein the clinic building is positioned on a first side of the at least one diagnosis bay, wherein the drive-thru medical facility further comprises a second building positioned on a second side of the at least one diagnosis bay that is opposite the first side of the at least one diagnosis bay.

14. A system comprising:
the drive-thru medical facility according to claim 1; and
at least one computing device, wherein the at least one computing device comprises at least one processor and at least one memory in communication with the at least one processor, wherein the at least one processor is configured to:
receive an input indicative of a diagnosis of a condition of a patient within a vehicle positioned within a diagnosis bay of the at least one diagnosis bay.

15. The system of claim 14, wherein the at least one processor is further configured to:
generate an invoice associated with examination of the patient; and
receive payment of the invoice.

16. The system of claim 14, wherein the at least one processor is further configured to store, in the at least one memory, data indicative of the diagnosis of the condition of the patient, wherein the stored data is associated with an account of the user.

17. A method of using the drive-thru medical facility of claim 1, the method comprising:
receiving, by at least one computing device, an input indicative of a diagnosis of a condition of a patient within a vehicle positioned within a diagnosis bay of the at least one diagnosis bay.

18. The method of claim 17, further comprising:
evaluating one or more physical properties of the patient; and
providing, to the computing device, the input indicative of the diagnosis of the condition of the patient.

19. The method of claim 18, further comprising:
conducting further evaluation of the patient within the clinic building.

20. The method of claim 18, further comprising receiving, by the at least one computing device, payment associated with evaluation and diagnosis of the condition of the patient.
